(19) United States
(12) Patent Application Publication
(10) Pub. No.: US 2012/0219885 A1
(43) Pub. Date: Aug. 30, 2012

(54) NOVEL NON-CRYSTALLIZING METHACRYLATES, PRODUCTION AND USE THEREOF

(75) Inventors: Thomas Fäcke, Leverkusen (DE); Friedrich-Karl Bruder, Krefeld (DE); Marc-Stephan Weiser, Leverkusen (DE); Thomas Rölle, Leverkusen (DE); Dennis Hönel, Zulpich (DE)

(73) Assignee: Bayer Intellectual Property GmbH, Monheim (DE)

(21) Appl. No.: 13/505,519

(22) PCT Filed: Nov. 2, 2010
(86) PCT No.: PCT/EP10/66633

§ 371 Date: May 2, 2012

(30) Foreign Application Priority Data
Nov. 3, 2009 (EP) 09013763.9

(51) Int. Cl. G03H 1/00; C07C 69/78; C07C 271/30; C07C 69/76

(52) U.S. Cl. 430/2; 560/10; 560/17; 560/28

(57) ABSTRACT

The invention relates to a novel non-crystallizing methacrylate and a method for the production thereof. The invention further relates to a photopolymer formulation comprising the methacrylate of the invention as well as to use of said photopolymer formulation for producing holographic media.

The invention relates to a novel, noncrystallizing methacrylate and a process for the preparation thereof. The invention furthermore relates to a photopolymer formulation comprising the methacrylate according to the invention and the use of the photopolymer formulation for the production of holographic media.

Photopolymers are materials which can be exposed by means of the superposition of two coherent light sources, resulting in the formation of a three-dimensional structure in the photopolymers which generally permits recording in the material by a regional change of the refractive index. Such structures are referred to as holograms, which can also be recorded as diffractive optical elements. The optical functions performed by such a hologram depend on the specific exposure.

WO 2008/125199 A1 describes a photopolymer formulation which contains polyurethane-based matrix polymers, an acrylate-based writing monomer and photoinitiators. In the cured state, the writing monomer and the photoinitiators are embedded with spatially isotropic distribution in the polyurethane matrix.

The acrylate writing monomers described in the PCT application are complicated to prepare, since they inevitably require a final distillation step for removing the solvent. This is problematic also because polymerization of the acrylates can occur thereby.

It was an object to provide a methacryate which is easily obtainable, has no tendency to crystallize and is readily soluble in polyurethane networks. Moreover, it should be able to polymerize readily and be capable of permitting recording holograms in corresponding photopolymer formulations, In particular, no complicated working-up procedures should be necessary in its preparation.

This object is achieved by a methacrylate of the general formulae (I) or (II) and mixtures thereof

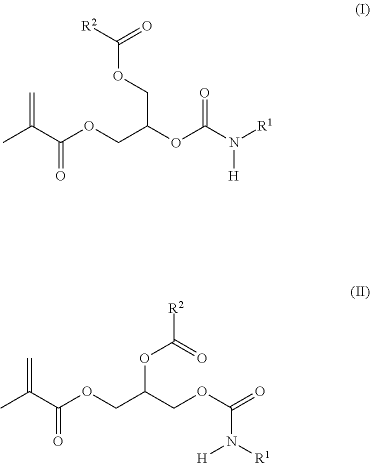

in which R¹ and R², independently of one another, are substituted phenyl radicals, substituted and/or unsubstituted naphthyl radicals.

Preferably, R¹ and/or R² may comprise 6-24 C atoms, 0-5 S atoms and 0-5 halogen atoms.

According to a preferred embodiment, R¹ and/or R² may be substituted by thioether groups, phenyl groups and/or halogen atoms.

It is very particularly preferable if R¹ and/or R² are naphthyl, 3-methylthiophenyl, 2-, 3- or 4-biphenyl, 2-bromophenyl.

The invention furthermore relates to a process for the preparation of a methacrylate according to the invention, in which an aromatic acid R²—COOH is reacted with glycidyl methacrylate and the product is then reacted with an aromatic isocyanate R¹—NCO.

The preparation of the methacrylates according to the invention is effected in a 2-stage synthesis. In the first reaction, an acid R²—COOH is reacted with glycidyl methacrylate, a mixture of two alcohols being formed according to reaction scheme 1.

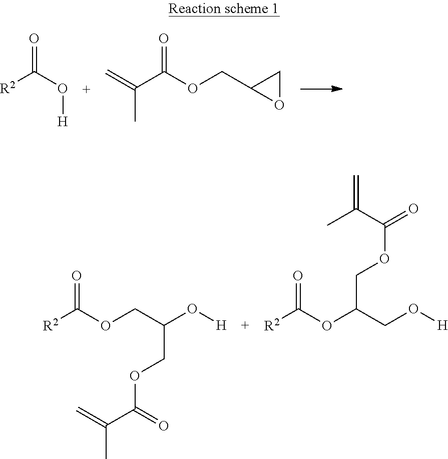

The reaction is typically effected at 20-180° C., preferably at 40-120° C. and particularly preferably at 50-100° C. Glycidyl methacrylate and a catalyst are initially introduced and the acid is added in portions. Owing to the limited solubility, the acid addition is determined by the stirrability of the batch. Progress of the reaction is indicated by the dissolution of the acid. The course of the reaction is monitored on the basis of the change in the epoxide content. ¹H-NMR spectroscopy is particularly suitable here as a detection method.

The reaction time can range from a few hours to days. Catalysts accelerate the reaction efficiently. Different classes of substance can be used as catalysts: for example, Broensted acids, such as phosphoric acid, phosphorous acid, sulphuric acid; Lewis acids, such as zinc acetate, zinc cetylacetonate, titanium(IV) methoxide, tetrakis(dimethylamino)zirconium, Lewis bases, such as 2-methylimidazoles, dimethylaminopyridine, borane pyridine complex, tris(dimethylamino)borane, triphenylphosphine, tris(o-tolyl)phosphine, choline chlorides, tris(4-dimethyleneaminophenyl)phosphine, tris(4-methoxyphenyl)phosphine, 1,4,5,6-tetrahydropyrimidine, diazabicycloundecane (DABCO) and other amines, and ammonium or phosphonium salts, such as, for example, tetraethylammonium trifluoroacetate, tetrabutyl-phosphonium bromide, benzyltrimethylammonium bromide, benzyltrimethylammonium chloride, tetrabutylphosphonium chloride and also tetrakis(dimethylamino)silane. Typically, between 0.01 and 1%, preferably 0.05-0.2% by weight, of the catalyst is used. Triphenylphosphine is preferably used.

In a second reaction step, the alcohol mixture is urethanized with a monoisocyanate R1-NCO to give the methacrylate mixture according to reaction scheme 2.

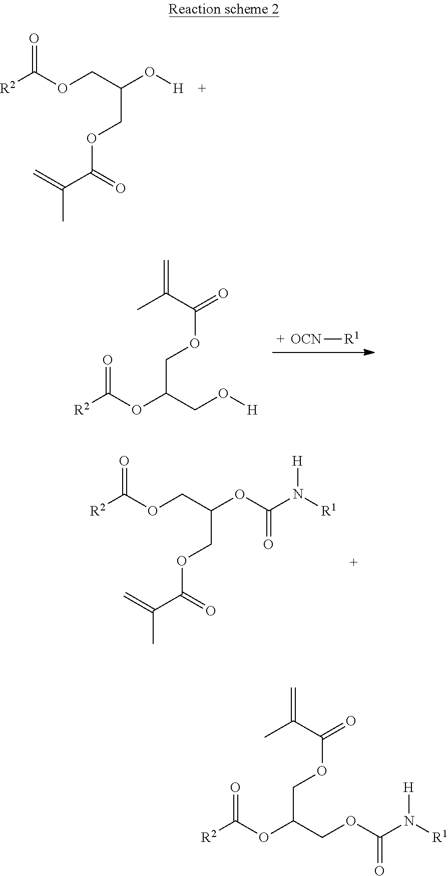

The urethanization is typically effected at 20-180° C., preferably at 40-120° C. and particularly preferably at 50-100° C. The alcohol is initially introduced as a product of the first stages, optionally together with a catalyst, and the isocyanate is then added dropwise.

The reaction is complete when the NCO content has fallen below 1%, preferably below 0.1% by weight. The NCO content can be determined by means of IR spectroscopy or by titration.

It is possible to separate the isomer mixture by customary methods known to the person skilled in the art. Preparative column chromatography is suitable for this purpose. The separation can be effected after the first stage or after the second stage.

It is also possible additionally to introduced the isocyanate and then to add the alcohol dropwise. The preferred method of addition will be influenced in the specific case by the handling and hence by the viscosity of the starting materials.

Catalysts which may be used for the reaction of reaction scheme 2 are amines and metal compounds of the metals tin, zinc, iron, bismuth, molybdenum, cobalt, calcium, magnesium and zirconium. Tin octanoate, zinc octonate, dibutyltin dilaurate, dimethyltin dicarboxylate, iron(III) acetylacetonate, iron(II) chloride, zinc chloride, tetraalkylammonium hydroxides, alkali metal hydroxides, alkali metal alcoholates, alkali metal salts of long-chain fatty acids having 10 to 20 carbon atoms and optionally OH side groups, lead octanoate or tertiary amines, such as triethylamine, tributylamine, dimethylbenzylamine, dicyclohexylmethylamine, dimethylcyclohexylamine, N,N,N′,N′-tetramethyldiaminodiethyl ether, bis(dimethylaminopropyl)urea, N-methyl- or N-ethylmorpholine, N,N′-dimorpholinodiethyl ether (DMDEE), N-cyclohexylmorpholine, N,N,N′,N′-tetramethyl-ethylenediamine, N,N,N′,N′-tetramethylbutanediamine, N,N,N′,N′-tetramethylhexane-1,6-diamine, pentamethyldiethylenetriamine, dimethylpiperazine, N-dimethylamino-ethylpiperidine, 1,2-dimethylimidazole, N-hydroxypropylimidazole, 1-azabicyclo[2.2.0]octane, 1,4-diazabicyclo[2.2.2]octane (Dabco), or alkanolamine compounds such as triethanolamine, triisopropanolamine, N-methyl- and N-ethyl-diethanolamine, dimethylaminoethanol, 2-(N,N-dimethylaminoethoxy)ethanol or N-tris(dialkylaminoalkyl)hexahydrotriazines, e.g. N,N′,N-tris(dimethylaminopropyl)-s-hexahydrotriazine, diazabicyclononane, diazabicycloundecane, 1,1,3,3-tetramethylguanidine, 1,3,4,6,7,8-hexahydro-1-methyl-2H-pyrimido(1,2-a)pyrimidine are preferred. Particularly preferred catalysts here are dibutyltin dilaurate, dimethyltin dicarboxylate, iron(III) acetylacetonate, 1,4-diazabicyclo[2.2.2]octane, diazabicyclononane, diazabicycloundecane, 1,1,3,3-tetramethylguanidine, 1,3,4,6,7,8-hexahydro-1-methyl-2H-pyrimido(1,2-a)pyrimidine.

During the synthesis, air is usually passed through in order to avoid an undesired polymerization. During this procedure, it must be ensured that sufficient phenols, such as, for example, p-methoxyphenol or ionol, are present, amounts between 0.001 and 0.1% by weight being sufficient. However, it is also possible to use other free radical stabilizers which are described in detail further below.

The isocyanates R1-NCO comprise monoisocyanates, it being possible for R1 to have the meanings mentioned above. The isomeric methylthiophenyl isocyanate, such as 2-methylthiophenyl isocyanate, 3-methylthiophenyl isocyanate, 4-methylthiophenyl isocyanate, bis-, tris-, tetra- and penta(methylthio)phenyl isocyanate, ethylthiophenyl isocyanate, n-propylthiophenyl isocyanate, isopropylthiophenyl isocyanate, butylthiophenyl isocyanate, phenylthiophenyl isocyanate, bis(phenylthio)phenyl isocyanate, naphtylthiophenyl isocyanate, biphenyl isocyanate, such as 2-biphenyl isocyanate, 3-biphenyl isocyanate and 4-biphenyl isocyanate, triphenyl isocyanates, chlorophenyl isocyanate, dichlorophenyl isocyanate, such as 3,4-dichlorophenyl isocyanate, tri-, tetra- and pentachlorophenyl isocyanate and mixtures thereof, bromophenyl isocyanate, di-, tri-, tetra- and pentabromophenyl isocyanate and mixtures thereof are particularly suitable. Mixed substituents on the phenyl isocyanate are also possible, such as, for example, chlorobromophenyl isocyanate, bromo(methylthio)phenyl isocyanate, methylthio(phenyl)phenyl isocyanate and analogues.

Substituted or unsubstituted naphthyl isocyanates are likewise suitable, such as naphthyl isocyanate, phenylnaphthyl isocyanate, thiomethylnaphthyl isocyanate, thioethylnaphthyl isocyanate, thiopropylnaphthyl isocyanate, bromonaphthyl isocyanate, chloronaphthyl isocyanate, and naphthyl isocyanates which are polysubstituted and those which are mixed substituents.

The isomeric biphenyl isocyanate, naphthyl isocyanate, the isomeric methylthiophenyl isocyanate, bromophenyl isocyanate, 3,4-dichlorophenyl isocyanate are preferred.

2-Biphenyl isocyanate, 3-biphenyl isocyanate and 4-biphenyl isocyanate, 3-methylthiophenyl isocyanate and napthyl isocyanate are particularly preferred.

Suitable acids R2-COOH are in particular aromatic acids, it being possible for these to be a substituted benzoic acid or a substituted or unsubstituted naphthylic acid. In the formula R2-COOH, R2 may have the abovementioned meanings.

Phenylbenzoic acids, such as 2-, 3- and 4-phenylbenzoic acid, and the isomeric bis- and tris-(phenyl)benzoic acids, the isomeric naphthylbenzoic acids, chlorobenzoic acid, dichlorobenzoic acid, trichlorobenzoic acid, tetrachlorobenzoic acid, pentachlorobenzoic acid, the isomeric bromobenzoic acids, dibromobenzoic acid, tribromobenzoic acid, tetrabromobenzoic acid, pentabromobenzoic acid, methylthiophenylbenzoic acid, 2-methylthiophenylbenzoic acid, 3-methylthiobenzoic acid, 4-methylthiobenzoic acid, bis-, tris-, tetra- and penta(methylthio)benzoic acid, ethylthiobenzoic acid, n-propylthiobenzoic acid, isopropylthiobenzoic acid, butylthiobenzoic acid, phenylthiobenzoic acid, bis(phenylthio)benzoic acid, naphthylthiobenzoic acid, can preferably be used.

2-, 3- and 4-phenylbenzoic acid, the isomeric naphthoic acids, the isomeric chlorobenzoic acids, the isomeric bromobenzoic acids, the isomeric methylthiobenzoic acids are particularly preferred.

2-, 3- and 4-phenylbenzoic acid, 2-bromobenzoic acid and 1-naphthoic acid are very particularly preferred.

The invention furthermore relates to a photopolymer formulation comprising matrix polymers, writing monomers and photoinitiators, the writing monomers comprising a methacrylate according to the invention.

Suitable matrix polymers are amorphous thermoplastics, such as polyacrylates, polymethyl methacrylates or copolymers of methyl methacrylate, methacrylic acid or other alkyl acrylates and alkyl methacrylates and acrylic acid; polyvinyl acetate and its partly hydrolysed derivatives, such as polyvinyl alcohols, gelatin, cellulose esters and cellulose ethers, such as cellulose acetobutyrate, and polyethylene oxides. The matrix polymers are particularly preferably polyurethanes.

Furthermore, matrix polymers based on a functional binder and on a crosslinking agent are also suitable. Two-component epoxy systems and urethane systems can be used for this purpose, two-component urethane systems being preferred. For the use of urethane crosslinking, a polyisocyanate crosslinking agent and a hydroxy- or amine-functional binder (resin) are required.

Suitable compounds of the polyisocyanate crosslinking agents are all aliphatic, cycloaliphatic, aromatic or araliphatic di- and triisocyanates known per se to the person skilled in the art, it being unimportant whether these were obtained by means of phosgenation or by phosgene-free processes. In addition, high molecular weight secondary products (oligo- and polyisocyanates) of monomeric di- and/or triisocyanates having a urethane, urea, carbodiimide, acylurea, isocyanurate, allophanate, biuret, oxadiazinetrione, uretdione or iminooxadiazinedione structure which are well known per se to the person skilled in the art can also be used, in each case individually or as any desired mixtures with one another.

Monomeric di- or triisocyanates, such as butylene diisocyanate, hexamethylene diisocyanate (HDI), isophorone diisocyanate (IPDI), trimethylhexamethylene diisocyanate (TMDI), 1,8-diisocyanato-4-(isocyanatomethyl)octane, isocyanatomethyl-1,8-octane diisocyanate (TIN), 2,4- and/or 2,6-toluoylene diisocyanate, are suitable. Likewise, the trimers of hexamethylene diisocyanate having an isocyanurate and/or iminooxadiazinetrione structure are also suitable.

The use of isocyanate-functional prepolymers having urethane, allophanate or biuret structures, as can be obtained in the manner well known per se by reacting the abovementioned di-, tri- or polyisocyanates in excess with hydroxy- or amino-functional compounds, is also possible. Any unconverted starting isocyanate can subsequently be removed in order to obtain products having a low monomer content. The use of catalysts well known per se to a person skilled in art from polyurethane chemistry may be helpful for accelerating the prepolymer formation.

Oligo- and polyisocyanates derived from monomeric diisocyanates having a urethane, urea, carbodiimide, acylurea, isocyanurate, allophanate, biuret, oxadiazinetrione, uretdione or iminooxadiazinedione structure, which are used in each case individually or as any desired mixtures with one another, are preferably suitable.

Oligo- and polyisocyanates of aliphatic diisocyanates having an isocyanurate, allophanate, biuret, uretdione or iminooxadiazinedione structure, which are used in each case individually or as any desired mixtures with one another, are particularly preferred.

Suitable hydroxy- or amine-functional binders (resins) are di- or polyols and/or -amines having a number average molecular weight in the range from 500 to 13000 g/mol, preferably 700 to 8500 g/mol.

Preferred resins for this purpose have an average functionality of 1.5 to 3.5, preferably of 1.8 to 3.2, particularly preferably 1.9 to 3.1.

Such polyols of the abovementioned type are, for example, polyester alcohols based on aliphatic, cycloaliphatic and/or aromatic di-, tri- and/or polycarboxylic acids with di-, tri-, and/or polyfunctional alcohols and lactone-based polyester alcohols.

Preferred polyester alcohols having a molecular weight of preferably 500 to 4000, particularly preferably 650 to 2500, g/mol are, for example, reaction products of adipic acid with hexanediol, butanediol or neopentyl glycol or mixtures of said diols.

Polyether polyols which are obtainable by polymerization of cyclic ethers or by reaction of alkylene oxides with a starter molecule are also suitable.

The polyethylene and/or polypropylene glycols having a number average molecular weight of 500 to 13000 g/mol and furthermore polytetrahydrofurans having a number average molecular weight of 500 to 8000, preferably of 650 to 3000 g/mol may be mentioned by way of example.

Preferred polyetherpolyols are polyethylene/polypropylene glycols having a polypropylene content of at least 70% and a functionality of 1.9 to 3.1.

Polyester-polyether-polyester block polyols, which can be obtained by reacting polyether polyols with lactones, are also suitable.

Polyester-polyether-polyester block polyols are preferred; polyester-polyether-polyester block polyols based on polytetrahydrofurans having a number average molecular weight of 200 to 2000 g/mol and ε-caprolactone are particularly preferred, these polyester-polyether-polyester block polyols having a number average molecular weight of 1000 to 8000 g/mol.

Hyroxyl-terminated polycarbonates which are obtainable by reacting diols or lactone-modified diols or bisphenols, such as, for example, bisphenol A, with phosgene or carbonic acid diesters, such as diphenyl carbonate or dimethyl carbonate, are also suitable.

The polymeric carbonates of 1,6-hexanediol having a number average molecular weight of 500 to 8000 g/mol and the carbonates of reaction products of 1,6-hexanediol with ε-caprolactone in the molar ratio of from 1 to 0.1 may be mentioned by way of example. Preferred carbonates are abovementioned polycarbonatediols having a number average molecular weight of from 650 to 3000 g/mol and based on 1,6-hexanediol and/or carbonates of reaction products of 1,6-hexanediol with ε-caprolactone in the molar ratio of from 1 to 0.33.

Hydroxyl-terminated polyamide alcohols and hydroxyl-terminated polyacrylatediols, e.g. Tegomer® BD 1000 (Tego GmbH, Essen, Germany) can also be used.

Polyethylene/polypropylene glycols having a polypropylene content of at least 70% and a functionality of 1.9 to 2.5 and polyester-polyether-polyester block polyols based on polytetrahydrofurans having a number average molecular weight of 400 to 1400 g/mol and ε-caprolactone are particularly preferred, these polyester-polyether-polyester block polyols having a number average molecular weight of 1500 to 4000 g/mol.

Photoinitiators are usually initiators which can be activated by actinic radiation and initiate polymerization of the corresponding polymerizable groups. Photoinitiators are commercially sold compounds known per se, a distinction being made between monomolecular (type I) and bimolecular (type II) initiators. Furthermore, these initiators are used for free radical, anionic (or), cationic (or mixed) forms of the abovementioned polymerizations, depending on their chemical nature. The photoinitiators can preferably comprise an anionic, cationic or neutral dye and a coinitiator.

(Type I) systems for free radical photopolymerization are, for example, aromatic ketone compounds, e.g. benzophenones in combination with tertiary amines, alkylbenzophenones, 4,4′-bis(dimethylamino)benzophenone (Michlers ketone), anthrone and halogenated benzophenones or mixtures of said types. (Type II) initiators, such as benzoin and its derivatives, benzil ketals, acylphosphine oxides, e.g. 2,4,6-trimethylbenzoyl-diphenylphosphine oxide, bisacylophosphine oxides, phenylglyoxylic acid esters, camphorquinone, alpha-aminoalkylphenones, alpha-,alpha-dialkoxyacetophenones, 1-[4-(phenylthio)phenyl]octane-1,2-dione 2-(O-benzoyloxime), differently substituted hexarylbisimidazoles (HABI) with suitable coinitiators such as, for example, mercaptobenzoxazole and alpha-hydroxyalkylphenones are also suitable. Photoinitiator systems described in EP-A 0223587, consisting of a mixture of an ammonium arylborate and one or more dyes, can also be used as photoinitiator. For example, tetrabutylammonium triphenylhexylborate, tetrabutylammonium triphenylbutylborate, tetrabutylammonium trinaphthylbutylborate, tetramethylammonium triphenylbenzylborate, tetra(n-hexyl)ammonium (sec-butyl)triphenylborate, 1-methyl-3-octylimidazolium dipentyldiphenylborate, tetrabutylammonium tris(4-tert-butyl)phenylbutylborate, tetrabutylammonium tris(3-fluorophenyl)hexylborate and tetrabutylammonium tris(3-chloro-4-methylphenyl)hexylborate are suitable as an ammonium arylborate. Suitable dyes are, for example, new methylene blue, thionine, basic yellow, pinacynol chloride, rhodamine 6G, gallocyanine, ethyl violet, victoria blue R, celestine blue, quinaldine red, crystal violet, brilliant green, astrazone orange G, darrow red, pyronine Y, basic red 29, pyrillium I, safranine 0, cyanine and methylene blue, azur A (Cunningham et al., RadTech'98 North America UV/EB Conference Proceedings, Chicago, Apr. 19-22, 1998).

The photoinitiators used for the anionic polymerization are as a rule (type I) systems and are derived from transition metal complexes of the first series. Here are chromium salts, such as, for example, trans-Cr(NH₃)₂(NCS)₄— (Kutal et al, Macromolecules 1991, 24, 6872) or ferrocenyl compounds (Yamaguchi et al. Macromolecules 2000, 33, 1152). A further possibility of anionic polymerization consists in the use of dyes, such as crystal violet leuconitrile or Malachite Green leuconitrile, which can polymerize cyanoacrylates by photolytic decomposition (Neckers et al. Macromolecules 2000, 33, 7761). However, the chromophore is incorporated into the polymer so that the resulting polymers are coloured throughout.

The photoinitiators used for the catinoic polymerization substantially comprise three classes: aryldiazonium salts, onium salts (here specifically: iodonium, sulphonium and selenonium salts) and organometallic compounds. On exposure to radiation both in the presence and in the absence of a hydrogen donor, phenyldiazonium salts can produce a cation which initiates the polymerization. The efficiency of the overall system is determined by the nature of the counterion used for the diazonium compound. The poorly reactive but very expensive SbF₆⁻, AsF₆⁻ or PF6⁻ is preferred here. These compounds are as a rule not very suitable for use in the coating of thin films since the surface quality is reduced (pinholes) by the nitrogen released after the exposure (Li et al., Polymeric Materials Science and Engineering, 2001, 84, 139). Onium salts, especially sulphonium or iodonium salts, are very widely used and also commercially avaialble in many forms. The photochemistry of these compounds has been investigated for a long time. The iodonium salts are initially decomposed homolytically after excitation and thus produce a free radical and a radical anion, which is stabilized by H abstraction and releases a proton and then initiates the cationic polymerization (Dektar et al. J. Org. Chem. 1990, 55, 639; J. Org. Chem., 1991, 56. 1838). This mechanism permits the use of iodonium salts also for free radical photopolymerization. Once again, the choice of the counterion is very important here; SbF₆⁻, AsF₆⁻ or PF₆⁻ is likewise preferred. Otherwise, the choice of the substitution of the aromatic is entirely free in this structure class and is determined substantially by the availability of suitable starting building blocks for the synthesis. The sulphonium salts are compounds which decompose according to Norrish(II) (Crivello et al., Macromolecules, 2000, 33, 825). In the case of the sulphonium salts too, the choice of the counterion is of critical importance, which manifests itself substantially in the curing rate of the polymers. The best results are as a rule obtained with SbF₆⁻ salts. Since the self-absorption of iodonium and sulphonium salts is at <300 nm, these compounds must be appropriately sensitized for the photopolymerization with near UV or short-wave visible light. This is effected by the use of more highly absorbing aromatics, such as, for example, anthracene and derivatives (Gu et al., Am. Chem. Soc. Polymer Preprints, 2000, 41 (2), 1266) or phenothiazine or derivatives thereof (Hua et al, Macromolecules 2001, 34, 2488-2494).

It may also be advantageous to use mixtures of these compounds. Depending on the radiation source used for the curing, type and concentration of photoinitiator must be adapted in a manner known to the person skilled in the art. Further details are described, for example, in P.K.T. Oldring (Ed.), Chemistry & Technology of UV & EB Formulations For Coatings, Inks & Paints, Vol. 3, 1991, SITA Technology, London, pages 61-328.

Preferred photoinitiators are mixtures of tetrabutylammonium triphenylhexylborate, tetrabutylammonium triphenylbutylborate, tetrabutylammonium trinaphthylbutylborate, tetrabutylammonium tris(4-tert-butyl)phenylbutylborate, tetrabutylammonium tris(3-fluorophenyl)hexylborate and tetrabutylammonium tris-(3-chloro-4-methylphenyl)-hexylborate with dyes, such as, for example, astrazone orange G, methylene blue, new methylene blue, azur A, pyrillium I, safranin O, cyanine, gallocyanine, brilliant green, crystal violet, ethyl violet and thionine.

Furthermore, in the formulations according to the invention, free radical stabilizers, catalysts, plasticizers and further additives can also be concomitantly used.

Suitable free radical stabilizers are inhibitors and antioxidants, as described in “Methoden der organischen Chemie [Methods of Organic Chemistry]” (Houben-Weyl), 4th edition, volume XIV/1, page 433ff, Georg Thieme Verlag, Stuttgart 1961. Suitable classes of substance are, for example, phenols, such as, for example, 2,6-di-tert-butyl-4-methylphenol, cresols, hydroquinones, benzyl alcohols, such as, for example, benzhydrol, optionally also quinones, such as, for example, 2,5-di-tert-butylquinone, optionally also aromatic amines, such as diisopropylamine or phenothiazine. Preferred free radical stabilizers are 2,6-di-tert-butyl-4-methylphenol, phenothiazine and benzhydrol.

Furthermore, one or more catalysts may be used. These preferably catalyze the urethane formation. These are in general the same catalysts which are also used in the second reaction stage in the preparation of the methacrylates according to the invention (see above).

For example solvents, plasticizers, levelling agents, wetting agents, antifoams or adhesion promoters, but also polyurethanes, thermoplastic polymers, oligomers, compounds having further functional groups, such as, for example, acetals, epoxide, oxetanes, oxazolines, dioxolanes, and/or hydrophilic groups, such as, for example, salts and/or polyethylene oxides may be present as further auxiliaries and additives.

Preferably used solvents are readily volatile solvents having good compatibility with the formulations essential to the invention, for example ethyl acetate, butyl acetate, acetone.

Plasticizers used are preferably liquids having good dissolution properties, low volatility and high boiling points. Suitable plasticizers are the compounds known in polyurethane chemistry, such as esters of aromatic acids, such as, for example, dibutyl phthalate, triisononyl trimellitate or diethylene glycol dibenzoate; the alkanesulphonic acid esters of phenol; esters of aliphatic acids, such as, for example, diisononyl cyclohexane-1,2-dicarboxylic acid, acetyltributyl citrate, dibutyl sebacate, polyesters of adipic acid or dibutyl adipate; acetic acid esters, such as, for example, glyceryl triacetate; esters of unsaturated acids, such as di(2-ethylhexyl) maleate; esters of phosphoric acid, such as, for example, tributoxyethyl phosphate; sulphonamides, such as, for example, N-butylbenzenesulphonamide; mineral oils, such as aromatic oils, naphthenic oils and paraffinic oils; vegetable oils, such as camphor, epoxidized soya oil or linseed oil, castor oil, and ethers of short-chain alcohols and ethers such as, for example, hexanediol dibutyl ether or triethylene glycol dimethyl ether.

The photopolymer formulation may additionally contain urethanes as plasticizers, it being possible for the urethanes to be substituted in particular by at least one fluorine atom. The urethanes may preferably have the general formula (5)

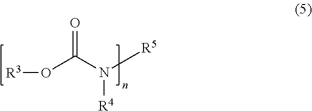

in which n≧1 and n≦8 and R³, R⁴, R⁵ are hydrogen and/or, independently of one another, linear, branched, cyclic or heterocyclic organic radicals which are unsubstituted or optionally also substituted by heteroatoms, preferably at least one of the radicals R³, R⁴, R⁵ being substituted by at least one fluorine atom and particularly preferably R³ being an organic radical having at least one fluorine atom.

It may also be advantageous simultaneously to use a plurality of additives of one type. Of course, it may also be advantageous to use a plurality of additives of a plurality of types.

In a further preferred embodiment, it is envisaged that the writing monomers additionally comprise a polyfunctional writing monomer, it being possible for this to be in particular a polyfunctional acrylate. The polyfunctional acrylate may have in particular the general formula (IV)

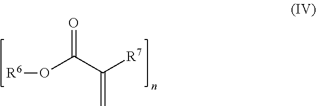

in which n≧2 and n≦4 and R⁶, R⁷ are hydrogen and/or, independently of one another, linear, branched, cyclic or heterocyclic organic radicals which are unsubstituted or optionally also substituted by heteroatoms.

It is also possible to add further unsaturated compounds, such as α,β-unsaturated carboxylic acid derivatives, such as acrylates, methacrylates, maleates, fumarates, maleimides, acrylamides, furthermore vinyl ethers, propenyl ethers, allyl ethers and compounds containing dicyclopentadienyl units and olefinically unsaturated compounds, such as, for example, styrene, α-methylstyrene, vinyltoluene, olefines, such as, for example, 1-octene and/or 1-decene, vinyl esters, (meth)acrylonitrile, (meth)acrylamide, methacrylic acid, acrylic acid. Acrylates and methacrylates are preferred.

In general, esters of acrylic acid or methacrylic acid are designated as acrylates or methacrylates, respectively. Examples of acrylates and methacrylates which can be used are methyl acrylate, methyl methacrylate, ethyl acrylate, ethyl methacrylate, ethoxyethyl acrylate, ethoxyethyl methacrylate, n-butyl acrylate, n-butyl methacrylate, tert-butyl acrylate, tert-butyl methacrylate, hexyl acrylate, hexyl methacrylate, 2-ethylhexyl acrylate, 2-ethylhexyl methacrylate, butoxyethyl acrylate, butoxyethyl methacrylate, lauryl acrylate, lauryl methacrylate, isobornyl acrylate, isobornyl methacrylate, phenyl acrylate, phenyl methacrylate, p-chlorophenyl acrylate, p-chlorophenyl methacrylate, p-bromophenyl acrylate, p-bromophenyl methacrylate, 2,4,6-trichlorophenyl acrylate, 2,4,6-trichlorophenyl methacrylate, 2,4,6-tribromophenyl acrylate, 2,4,6-tribromophenyl methacrylate, pentachlorophenyl acrylate, pentachlorophenyl methacrylate, pentabromophenyl acrylate, pentabromophenyl methacrylate, pentabromobenzyl acrylate, pentabromobenzyl methacrylate, phenoxyethyl acrylate, phenoxyethyl methacrylate, phenoxyethoxyethyl acrylate, phenoxyethoxyethyl methacrylate, phenylthioethyl acrylate, phenylthioethyl methacrylate, 2-naphthyl acrylate, 2-naphthyl methacrylate, 1,4-bis(2-thionaphthyl)-2-butyl acrylate, 1,4-bis(2-thionaphthyl)-2-butyl methacrylate, propane-2,2-diylbis[(2,6-dibromo-4,1-phenylen)oxy(2-{[3,3,3-tris(4-chlorophenyl)propanoyl]oxy}propane-3,1-diyl)oxyethane-2,1-diyl]diacrylate, bisphenol A diacrylate, bisphenol A dimethacrylate, tetrabromobisphenol A diacrylate, tetrabromobisphenol A dimethacrylate and the ethoxylated analogue compounds thereof, N-carbazolyl acrylates, to mention but a selection of acrylates and methacrylates which can be used.

Of course, further urethane acrylates can also be used. Urethane acrylates are understood as meaning compounds having at least one acrylic acid ester group and which additionally have at least one urethane bond. It is known that such compounds can be obtained by reacting a hydroxy-functional acrylic acid ester with an isocyanate-functional compound.

Examples of isocyanates which can be used for this purpose are aromatic, araliphatic, aliphatic and cycloaliphatic di-, tri- or polyisocyanates. It is also possible to use mixtures of such di-, tri- or polyisocyanates. Examples of suitable di-, tri- or polyisocyanates are butylene diisocyanate, hexamethylene diisocyanate (HDI), isophorone diisocyanate (IPDI), 1,8-diisocyanato-4-(isocyanatomethyl)octane, 2,2,4- and/or 2,4,4-trimethylhexamethylene diisocyanate, the isomeric bis(4,4′-isocyanatocyclohexyl)methanes and mixtures thereof having any desired isomer content, isocyanatomethyl-1,8-octane diisocyanate, 1,4-cyclohexylene diisocyanate, the isomeric cyclohexanedimethylene diisocyanates, 1,4-phenylene diisocyanate, 2,4- and/or 2,6-toluoylene diisocyanate, 1,5-naphthylene diisocyanate, 2,4′- or 4,4′-diphenylmethane diisocyanate, 1,5-naphthylene diisocyanate, m-methylthiophenyl isocyanate, triphenylmethane 4,4′,4″-triisocyanate and tris(p-isocyanatophenyl) thiophosphate or derivatives thereof having a urethane, urea, carbodiimide, acylurea, isocyanurate, allophanate, biuret, oxadiazinetrione, uretdione, or iminooxadiazinedione structure and mixtures thereof. Aromatic or araliphatic di-, tri- or polyisocyanates are preferred.

Suitable hydroxy-functional acrylates or methacrylates for the preparation of urethane acrylates are, for example, compounds such as 2-hydroxyethyl (meth)acrylate, polyethylene oxide mono(meth)acrylates, polypropylene oxide mono(meth)acrylates, polyalkylene oxide mono(meth)acrylates, poly(ε-caprolactone) mono(meth)acrylates, such as, for example, Tone® M100 (Dow, Schwalbach, Germany), 2-hydroxypropyl (meth)acrylate, 4-hydroxy-butyl (meth)acrylate, 3-hydroxy-2,2-dimethylpropyl (meth)acrylate, hydroxypropyl (meth)acrylate, 2-hydroxy-3-phenoxypropyl acrylate, the hydroxy-functional mono-, di- or tetraacrylates of polyhydric alcohols, such as trimethylolpropane, glycerol, pentaerythritol, dipentaerythritol, ethoxylated, propoxylated or alkoxylated trimethylolpropane, glycerol, pentaerythritol, dipentaerythritol or industrial mixtures thereof. 2-Hydroxyethyl acrylate, hydroxypropyl acrylate, 4-hydroxybutyl acrylate and poly(ε-caprolactone) mono(meth)acrylates are preferred. In addition, isocyanate-reactive oligomeric or polymeric unsaturated compounds containing acrylate and/or methacrylate groups, alone or in combination with the abovementioned monomeric compounds, are suitable. The epoxy (meth)acrylates known per se, containing hydroxyl groups and having OH contents of 20 to 300 mg KOH/g or polyurethane (meth)acrylates containing hydroxyl groups and having OH contents of 20 to 300 mg KOH/g or acrylated polyacrylates having OH contents of 20 to 300 mg KOH/g and mixtures thereof with one another and mixtures with unsaturated polyesters containing hydroxyl groups and mixtures with polyester (meth)acrylates or mixtures of unsaturated polyesters containing hydroxyl groups with polyester (meth)acrylates can also be used.

In particular, urethane acrylates obtainable from the reaction of tris(p-isocyanatophenyl) thiophosphate and m-methylthiophenyl isocyanate with alcohol-functional acrylates, such as hydroxyethyl (meth)acrylate, hydroxypropyl (meth)acrylate and hydroxybutyl (meth)acrylate, are preferred.

The invention furthermore relates to the use of a photopolymer formulation according to the invention for the production of holographic media which can be processed by appropriate exposure processes for optical applications in the total visible and near UV range (300-800 nm) to give holograms. Visual holograms comprise all holograms which can be recorded by methods known to the person skilled in the art. These include, inter alia, in-line (Gabor) holograms, off-axis holograms, full-aperture transfer holograms, white light transmission holograms (“rainbow holograms”), Denisyuk holograms, off-axis reflection holograms, edge-lit holograms and holographic stereograms; reflection holograms, Denisyuk holograms and transmission holograms are preferred. Possible optical functions of the holograms which can be produced with the photopolymer compositions according to the invention may correspond to the optical functions of light elements such as lenses, mirrors, deflection mirrors, filters, diffuser screens, diffraction elements, light conductors, waveguides, projection screens and/or masks. Frequently, these optical elements show a frequency selectivity, depending on how the holograms were exposed and on the dimensions of the hologram.

In addition, holographic images or representations, such as, for example, for personal portraits, biometric representations in security documents or generally of images or image structures for advertising, security labels, trademark protection, trademark branding, labels, design elements, decorations, illustrations, multi-journey tickets, images and the like and images which can represent digital data, inter alia also in combination with the products described above, can also be produced by means of the photopolymer compositions according to the invention. Holographic images may give the impression of a three-dimensional image, but they may also represent image sequences, short films or a number of different objects, depending on the angle from which they are illuminated, the light source (including moving light source) with which they are illuminated, etc. Owing to these various design possibilities, holograms, in particular volume holograms, are an attractive technical solution for the abovementioned application.

The photopolymer formulation can be used in particular as a holographic medium in the form of a film. A layer of a material or material composite which is transparent to light in the visible spectral range (transmission greater than 85% in the wavelength range of 400 to 780 nm), as a support, is coated on one or both sides and optionally a covering layer is applied to the photopolymer layer or layers.

Preferred materials or material composites of the support are based on polycarbonate (PC), polyethylene terephthalate (PET), polybutylene terephthalate, polyethylene, polypropylene, cellulose acetate, cellulose hydrate, cellulose nitrate, cycloolefin polymers, polystyrene, polyepoxides, polysulphone, cellulose triacetate (CTA), polyamide, polymethyl methacrylate, polyvinyl chloride, polyvinyl butyral or polydicyclopentadiene or mixtures thereof. They are particularly preferably based on PC, PET and CTA. Material composites may be film laminates or coextrudates. Preferred material composites are duplex and triplex films based on one of the schemes A/B, A/B/A or A/B/C. PC/PET, PET/PC/PET and PC/TPU (TPU=thermoplastic polyurethane) are particularly preferred.

As an alternative to the abovementioned plastic supports, it is also possible to use planar glass plates, which are used in particular for large-area exposures with accurate imaging, for example for holographic lithography [Ng, Willie W.; Hong, Chi-Shain; Yariv, Amnon Holographic interference lithography for integrated optics. IEEE Transactions on Electron Devices (1978), ED-25(10), 1193-1200. ISSN:0018-9383].

The materials or material composites of the support may be provided on one or both sides with an antiadhesive, antistatic, water-repellent or hydrophilized treatment. On the side facing the photopolymer layer, said modifications serve the purpose of enabling the photopolymer layer to be detached from the support without destruction. A modification of that side of the support which faces away from the photopolymer layer serves for ensuring that the media according to the invention meet specific mechanical requirements, which are required, for example, when processing in roll laminators, in particular in roll-to-roll methods.

### EXAMPLES

The invention will be explained in more detail below with reference to examples.

### Measurement of the Holographic Properties DE and Δn of the Holographic Media by Means of Two-Beam Interference in Reflection Arrangement

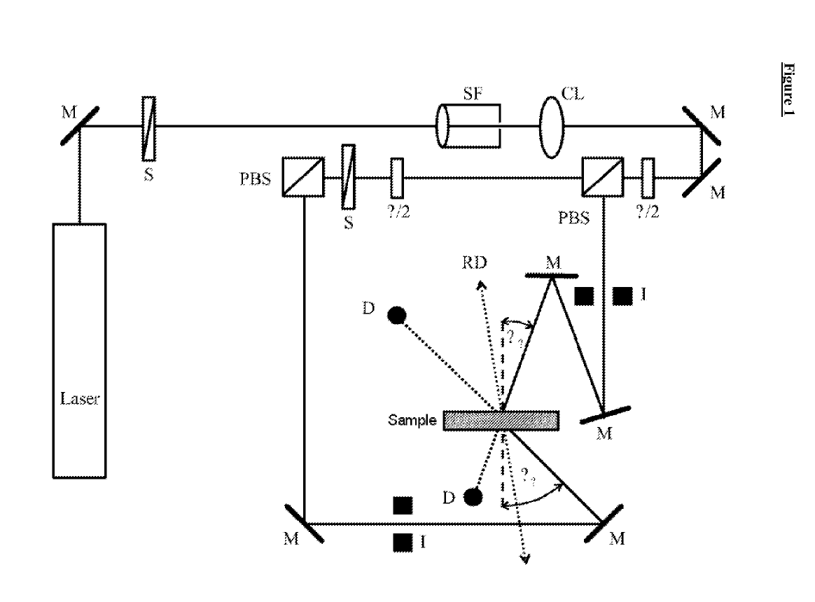

The holographic media which can be produced in this manner were then tested with regard to their holographic properties by means of a measuring arrangement according to FIG. 1, as follows:

the beam of an He—Ne laser (emission wavelength 633 nm) was conducted with the aid of the spatial filter (SF) and together with the collimation lens (CL) into a parallel homogeneous beam. The final cross sections of the signal and reference beam are established by the iris diaphragms (I). The diameter of the iris diaphragm opening is 0.4 cm. The polarization-dependent beam splitters (PBS) split the laser beam into two coherent equally polarized beams. By the λ/2 plates, the power of the reference beam was adjusted to 0.5 mW and the power of the signal beam to 0.65 mW. The powers were determined using the semiconductor detectors (D) with the sample removed. The angle of incidence (α₀) of the reference beam is −21.8° and the angle of incidence (β₀) of the signal beam is 41.8°. The angles are measured starting from the sample normal to the beam direction. According to FIG. 1, α₀ therefore has a negative sign and β₀ a positive sign. At the location of the sample (medium), the interference field of the two overlapping beams produced a grating of light and dark strips which are perpendicular to the angle bisectors of the two beams incident on the sample (reflection hologram). This strip spacing Λ, also referred to as grating period, in the medium is ˜225 nm (the refractive index of the medium assumed to be ˜1.504).

FIG. 1 shows the holographic experimental setup with which the diffraction efficiency (DE) of the media was measured. FIG. 1 shows the geometry of a Holographic Media Tester (HMT) at λ=633 nm (He—Ne laser): M=mirror, S=shutter, SF=spatial filter, CL=collimator lens, λ/2=λ/2 plate, PBS=polarization-sensitive beam splitter, D=detector, I=iris diaphragm, α₀=−21.8°, β₀=41.8° are the angles of incidence of the coherent beams, measured outside the sample (outside the medium). RD=reference direction of the turntable.

Holograms were recorded in the medium in the following manner:
both shutters (S) are opened for the exposure time t.
thereafter, with closed shutters (S), the medium was allowed a time of 5 minutes for diffusion of the as yet unpolymerized writing monomers.

The holograms recorded were read in the following manner. The shutter of the signal beam remained closed. The shutter of the reference beam was opened. The iris diaphragm of the reference beam was closed to a diameter of <1 mm. This ensured that the beam was always completely in the previously recorded hologram for all angles of rotation (Ω) of the medium. The turntable, under computer control, covered the angle range from Ωₘᵢₙ to Ωₘₐₓ with an angle step width of 0.05°. Ω is measured from the sample normal to the reference direction of the turntable. The reference direction of the turntable is obtained when the angle of incidence of the reference beam and that of the signal beam has the same absolute value on recording of the hologram, i.e. α₀=−31.8° and β₀=31.8°. Ω_{recording}=0°. For α₀=−21.8° and β₀=41.8°, Ω_{recording} is therefore 10°. In general, the following is true for the interference field during recording of the hologram:

α₀θ₀+Ω_{recording}.

θ₀ is the semiangle in the laboratory system outside the medium and the following is true during recording of the hologram:

$${θ}_{0} = \frac{{α}_{0} - {β}_{0}}{2} .$$

In this case, θ₀ is therefore −31.8°. At each angle of rotation Ω approached, the powers of the beam transmitted in zeroth order were measured by means of the corresponding detector D and the powers of the beam diffracted in the first order were measured by means of the detector D. The diffraction efficiency was obtained at each angle Ω approached as the quotient of:

$$η = \frac{{P}_{D}}{{P}_{D} + {P}_{T}}$$

P_{D} is the power in the detector of the diffracted beam and P_{T} is the power in the detector of the transmitted beam.

By means of the method described above, the Bragg curve (describes the diffraction efficiency η as a function of the angle of rotation Ω of the recorded hologram) was measured and was stored in a computer. In addition, the intensity transmitted in the zeroth order was plotted against the angle of rotation Ω and stored in a computer.

The maximum diffraction efficiency (DE=ηₘₐₓ) of the hologram, i.e. its peak value, was determined at Ω_{reconstruction}. It may have been necessary for this purpose to change the position of the detector of the diffracted beam in order to determine this maximum value.

The refractive index contrast Δn and the thickness d of the photopolymer layer was now determined by means of the coupled wave theory (see H. Kogelnik, The Bell System Technical Journal, Volume 48, November 1969, Number 9 page 2909-page 2947) from the measured Bragg curve and the variation of the transmitted intensity as a function of angle. It should be noted that, owing to the thickness shrinkage due to the photopolymerization, the strip spacing Λ′ of the hologram and the orientation of the strips (slant) may differ from the strip spacing Λ of the interference pattern and the orientation thereof. Accordingly, the angle α₀′ or the corresponding angle of the turntable Ω_{reconstruction}, at which maximum diffraction efficiency is reached, will also differ from α₀ or from the corresponding Ω_{recording}, respectively. The Bragg condition changes as a result of this. This change is taken into account in the evaluation method. The evaluation method is described below:

All geometrical quantities which relate to the recorded hologram and not to the interference pattern are shown as quantities represented by dashed lines.

According to Kogelnik, the following is true for the Bragg curve η(Ω) of a reflexion hologram:

$$η = \{ \begin{matrix}\frac{1}{1 - \frac{1 - {( ξ / v )}^{2}}{{sin}^{2} ( \sqrt{{ξ}^{2} - {v}^{2}} )}} , & for {v}^{2} - {ξ}^{2} < 0 \\ \frac{1}{1 + \frac{1 - {( ξ / v )}^{2}}{{sinh}^{2} ( \sqrt{{v}^{2} - {ξ}^{2}} )}} , & for {v}^{2} - {ξ}^{2} \geq 0\end{matrix}$$

with:

$$v = \frac{π \cdot Δ n \cdot {d}^{′}}{λ \cdot \sqrt{\vert {c}_{s} \cdot {c}_{r} \vert}}$$

$$ξ = - \frac{{d}^{′}}{2 \cdot {c}_{s}} \cdot DP$$

$${c}_{s} = cos ( {ϑ}^{′} ) - cos ( {ψ}^{′} ) \cdot \frac{λ}{n \cdot {Λ}^{′}}$$

$${c}_{r} = cos ( {ϑ}^{′} )$$

$$DP = \frac{π}{{Λ}^{′}} \cdot ( 2 \cdot cos ( {ψ}^{′} - {ϑ}^{′} ) - \frac{λ}{n \cdot {Λ}^{′}} )$$

$${ψ}^{′} = \frac{{β}^{′} + {α}^{′}}{2}$$

$${Λ}^{′} = \frac{λ}{2 \cdot n \cdot cos ( {ψ}^{′} - {α}^{′} )}$$

On reading of the hologram (“reconstruction”), the following is true as described analogously above:

θ′₀=θ₀+Ω

sin(θ′₀)=n·sin(θ′)

Under the Bragg condition, the “dephasing” DP=0. Accordingly, the following is true:

α′₀=θ₀+Ω_{reconstruction}

sin(α′₀)=n·sin(α′)

The still unknown angle β′ can be determined from the comparison of the Bragg condition of the interference field during recording of the hologram and the Bragg condition during reading of the hologram, assuming that only thickness shrinkage takes place. The following is then true:

$$sin ( {β}^{′} ) = \frac{1}{n} \cdot [ sin ( {α}_{0} ) + sin ( {β}_{0} ) - sin ( {θ}_{0} + {Ω}_{reconstruction} ) ]$$

v is the grating thickness, ξ is the detuning parameter and ψ′ is the orientation (slant) of the refractive index grating which was recorded. α′ and β′ correspond to the angles α₀ and β₀ of the interference field during recording of the hologram, but measured in the medium and applicable to the grating of the hologram (after thickness shrinkage). n is the mean refractive index of the photopolymer and was set at 1.504. λ is the wavelength of the laser light in vacuo.

The maximum diffraction efficiency (DE=ηₘₐₓ) is then obtained for ξ=0 as:

$$D E = {tanh}^{2} ( v ) = {tanh}^{2} ( \frac{π \cdot Δ n \cdot {d}^{′}}{λ \cdot \sqrt{cos ( {α}^{′} ) \cdot cos ( {α}^{′} - 2 ψ )}} )$$

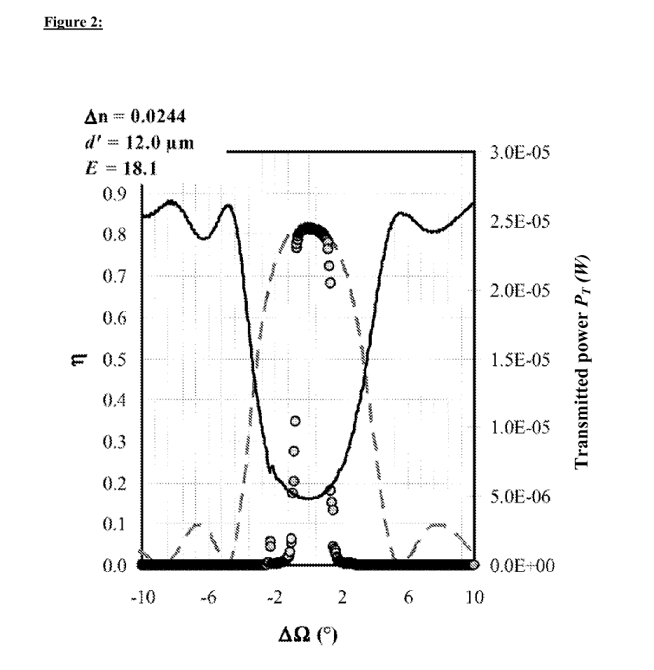

The measured data of the diffraction efficiency, the theoretical Bragg curve and the transmitted intensity are, as shown in FIG. 2, plotted against the centred angle of rotation ΔΩ≡Ω_{reconstruction}−Ω=α′₀−θ′₀, also referred to as angle detuning.

Since DE is known, the shape of the theoretical Bragg curve according to Kogelnik is determined only by the thickness d′ of the photopolymer layer. An is subsequently corrected via DE for a given thickness d′ so that measurement and theory of DE always agree. d′ is now adapted until the angular positions of the first secondary minima of the theoretical Bragg curve agree with the angular positions of the first secondary maxima of the transmitted intensity and additionally the full width at half maximum (FWHM) for the theoretical Bragg curve and for the transmitted intensity agree.

Since the direction in which a reflection hologram concomitantly rotates on reconstruction by means of an Ω scan, but the detector for the refracted light can detect only a finite angle range, the Bragg curve of broad holograms (small d′) is not completely detected in an Ω scan, but only the central region, with suitable detector positioning. The shape of the transmitted intensity which is complementary to the Bragg curve is therefore additionally used for adapting the layer thickness d′.

FIG. 2 shows the plot of the Bragg curve η according to the coupled wave theory (dashed line), of the measured diffraction efficiency (solid circles) and of the transmitted power (black solid line) against the angle detuning ΔΩ. FIG. 2 shows the measured transmitted power P_{T} (right y axis) as a solid line plotted against the angle detuning ΔΩ, the measured diffraction efficiency η (left y axis) as solid circles plotted against the angle detuning ΔΩ (if permitted by the finite size of the detector) and the adaptation of the Kogelnik theory as a dashed line (left y axis).

For a formulation, this procedure was possibly repeated several times for different exposure times t on different media, in order to determine the average energy dose of the incident laser beam at which DE reaches the saturation value during recording of the hologram. The average energy dose E is obtained from the powers of the two part-beams coordinated with the angles α₀ and β₀ (reference beam with Pᵣ=0.50 mW and signal beam with Pₛ=0.63 mW), the exposure time t and the diameter of the iris diaphragm (0.4 cm), as follows:

$$E ( mJ \text{/} {cm}^{2} ) = \frac{2 \cdot [ {P}_{r} + {P}_{s} ] \cdot t ( s )}{π \cdot {0.4}^{2} {cm}^{2}}$$

The powers of the part-beams were adapted so that the same power density is achieved in the medium at the angles α₀ and β₀ used.

### Preparation of the Methacrylates According to the Invention

### Example 1.1-1.3

### General Preparation Method According to Table 1

Glycidyl methacrylate, triphenylphosphine and ionol (2,5-di-tert-butyl-4-methylphenol) are initially introduced into a three-necked flask having a stirrer and reflux condenser and air is slowly passed through. Heating to 70° C. is effected. The acid is now added and stirring is continued under the stated conditions until the evaluation of the ¹H-NMR spectrum shows that the batch is substantially free of epoxide (when present, epoxide shows the characteristic resonances at ¹H-NMR (400 MHz, CDCl₃): δ=2.6 (dd), 2.8 (dd), 3.2 (m)).

### Example 2.1-2.3

### General Preparation Method According to Table 2

The product from the example shown in Table 2 and dibutyltin dilaurate are initially introduced at 60° C. into a three-necked flask having a stirrer and reflux condenser and air is slowly passed through. Within 25 minutes, the m-methylthiophenyl isocyanate is now added dropwise, an exothermic reaction taking place. Stirring is effected according to the stated reaction conditions and the product is obtained.

### Example 3.1-3.3

### General Preparation Method According to Table 3

The product from the example shown in Table 3 and dibutyltin dilaurate (DBTL) are initially introduced at 60° C. into a three-necked flask having a stirrer and reflux condenser and air is slowly passed through. Within 25 minutes, the naphthyl isocyanate is now added dropwise, an exothermic reaction taking place. Stirring is effected according to the stated reaction conditions and the product is obtained.

TABLE 1

| | | | Reaction | |
|---|---|---|---|---|
| Example | Product | Starting material | conditions | Description |
| 1.1 | 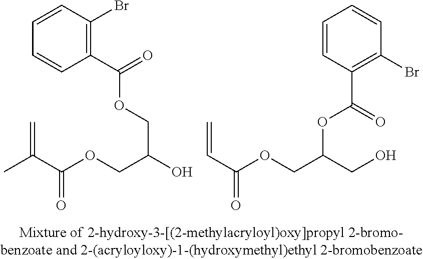 | 1.) 15.6 g of glycidyl methacrylate 2.) 72 mg of triphenylphosphine 3.) 0.4 mg of ionol 4.) 22.1 g of 2-bromobenzoic acid | 70° C., 42 h | clear, colourless liquid |
| 1.2 | 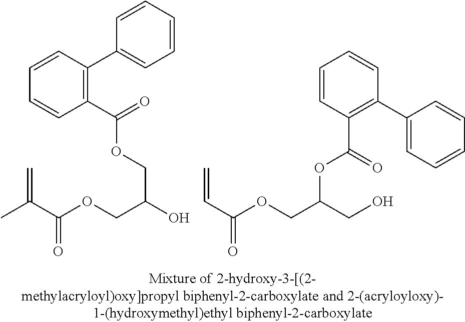 | 1.) 21.3 g of glycidyl methacrylate 2.) 98 mg of triphenylphosphine 3.) 15.3 mg of ionol 4.) 29.7 g of 2-phenylbenzoic acid | 70° C., 52 h | slightly yellowish, clear, medium- viscosity liquid |
| 1.3 | 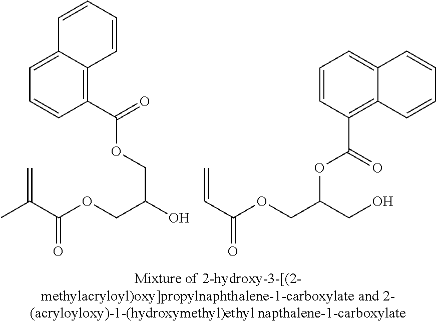 | 1.) 23.3 g of glycidyl methacrylate 2.) 107 mg of triphenylphosphine 3.) 15.4 mg of ionol 4.) 28.2 g of 1-naphthoic acid | 70° C., 44 h | clear, yellowish, viscous liquid |

TABLE 2

| | | | Reaction | |
|---|---|---|---|---|
| Example | Product | Starting material | conditions | Description |
| 2.1 | 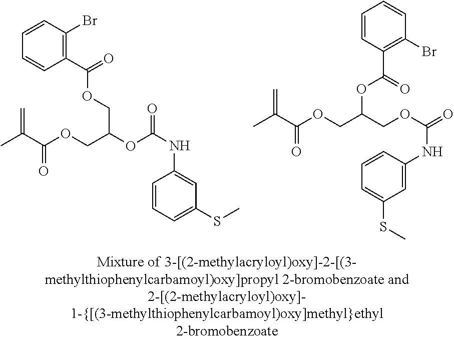 | 1.) 7.9 g of Example 1.1 2.) 1 mg of DBTL 3.) 3.8 g of m- methylthiophenyl isocyanate | 60° C., 22 h | clear, cream- coloured, highly viscous liquid |
| 2.2 | 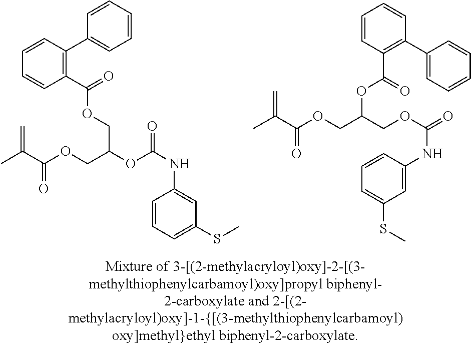 | 1.) 7.9 g of Example 1.2 2.) 2.0 mg of DBTL 3.) 5.0 g of m- methylthiophenyl isocyanate | 60° C., 19 h | clear, yellow, pasty mass |
| 2.3 | 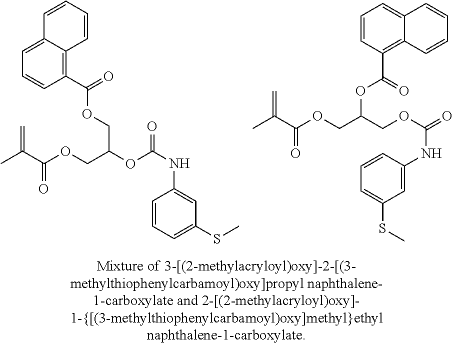 | 1.) 9.4 g of Example 1.3 2.) 1.0 mg of DBTL 3.) 5.0 g of m- methylthiophenyl isocyanate | 60° C., 22 h | highly viscous, slightly cloudy liquid |

TABLE 3

| | | | Reaction | |
|---|---|---|---|---|
| Example | Product | Starting material | conditions | Description |
| 3.1 | 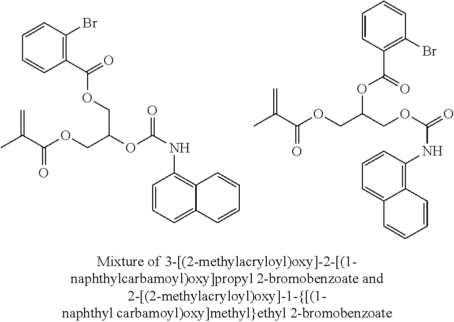 | 1.) 7.9 g of Example 1.1 2.) 1.0 mg of DBTL 3.) 3.9 g of 1-naphthyl isocyanate | 60° C., 22 h | cloudy, crea,- coloured highly viscous mass |
| 3.2 | 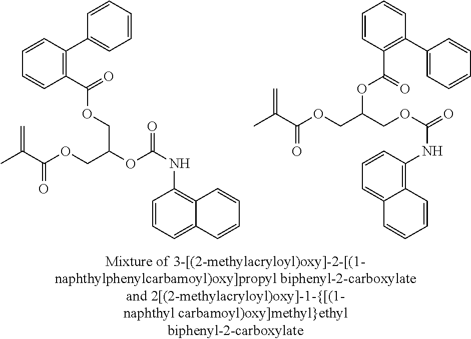 | 1.) 10.2 g of Example 1.2 2.) 2.0 mg of DBTL 3.) 5.1 g of 1-naphthyl isocyanate | 60° C., 19 h | cloudy, brownish glass |
| 3.3 | 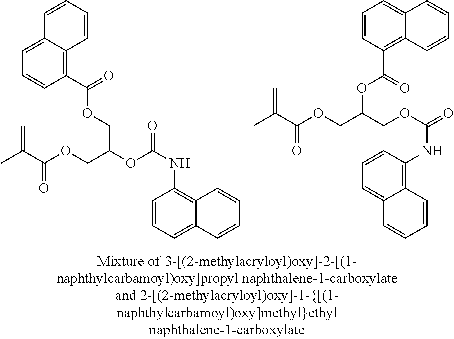 | 1.) 5.9 g of Example 1.3 2.) 1.0 mg of DBTL 3.) 3.2 g of 1-naphthyl isocyanate | 60° C., 21.5 h | cloudy, brownish glass |

### Preparation of the Polyol

### Example 4.0

In a 1 l flask, 18 g of zinc octanoate, 374.8 g of ε-caprolactone and 374.8 g of a difunctional polytetrahydrofuran polyetherpolyol (equivalent weight 500 g/mol OH, e.g. Terathane® 1000, a product of BASF SE, Ludwigshafen DE) are initially introduced and heated to 120° C. and kept at this temperature until the solids content was 99.5% by weight or higher (proportion of non-volatile constituents determined by storage of one gram of the product in an uncoated oven cover for one hour at 125° C., calculated according to the gravimetric results: final weight [g]·100/weight taken [g]=% by weight of solid). Thereafter, cooling was effected and the product was obtained as a waxy solid.

### Production of the Holographic Media

### Examples 5.1-5.6

5.927 g of the polyol component prepared as described above (Example 4.0) were mixed with 2.50 g of the product from Example 2.1, 0.10 g of CGI-909 (tetrabutylammonium tris(3-chloro-4-methylphenyl)(hexyl)borate, [1147315-11-4]), an experimental product released by CIBA Inc., Basle, Switzerland, 0.015 g of 20 μm glass beads (Whitehouse Scientific Ltd, Waverton, Chester, CH3 7PB, United Kingdom), 0.010 g of new methylene blue at 60° C. and 0.35 g of N-ethylpyrilidone so that a clear solution was obtained. Thereafter, cooling to 30° C. was effected, 1.098 g of Desmodur® XP 2410 (experimental product of Bayer MaterialScience AG, Leverkusen, Germany, hexane diisocyanate-based polyisocyanate, proportion of iminooxadiazine dione at least 30%, NCO content: 23.5%) were added and mixing was effected again. Finally, 0.006 g of Fomrez UL 28 (urethanization catalyst, commercial product of Momentive Performance Chemicals, Wilton, Conn., USA) was added and mixing was effected again briefly (by means of a Speedmixer). The liquid material obtained was then poured onto a glass plate and covered there with a second glass plate. The curing of the PU formulation takes places under 15 kg weights over several hours (usually overnight). A dimensionally stable glass sandwich (coupon) is obtained. Since different formulations having different starting viscosity and different curing rate of the matrix do not always lead to the same layer thicknesses d′ of the photopolymer layer, d′ is determined separately on the basis of the characteristics of the recorded holograms for each sample.

The media 5.2-5.6 were produced in an analogous manner from the examples listed in Tables 2 and 3.

TABLE 4
Results of the holographic testing of the methacrylates according to the invention as writing
monomer in the photopolymers according to the invention.

| R²\R¹ | 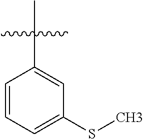 | 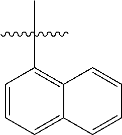 |
|---|---|---|
| 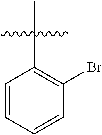 | Example 5.1 methacrylate from Example 2.1 Dn = 0.0026 exposure time 4 s energy dose 18.22 mJ/cm² layer thickness 26.4 μm | Example 5.4 methacrylate from Example 3.1 Dn = 0.0049 exposure time 1 s energy dose 4.56 mJ/cm² layer thickness 30.0 μm |
| 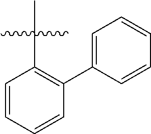 | Example 5.2 Methacrylate from Example 2.2 Dn = 0.0063 Exposure time 1 s Energy dose 4.56 mJ/cm² Layer thickness 20.5 μm | Example 5.5 Methacrylate from Example 3.2 Dn = 0.0099 Exposure time 1 s Energy dose 4.56 mJ/cm² Layer thickness 16.0 μm |
| 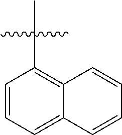 | Example 5.3 Methacrylate from Example 2.3 Dn = 0.0080 Exposure time 1 s Energy dose 4.46 mJ/cm² Layer thickness 17.0 μm | Example 5.6 Methacrylate from Example 3.3 Dn = 0.0094 Exposure time 1 s Energy dose 4.56 mJ/cm² Layer thickness 11.5 μm |

### Example 6.0

### Preparation of a fluorinated plasticizer [bis(2,2,3,3,4,4,5,5,6,6,7,7-dodecafluoroheptyl)-(2,2,4-trimethylhexane-1,6-diyl)biscarbamate]

In a three-necked round-bottomed flask with reflux condenser and stirrer, 0.02 g of Desmorapid Z (dibutyltin dilaurate) and 3.60 g of 2,4,4-trimethylhexane-1,6-diisocyanate (TMDI) were initially introduced and heated to 70° C. Thereafter, 11.39 g of 1H,1H-7H-perfluoroheptan-1-ol were added dropwise and the mixture was further kept at 70° C. until the isocyanate content had fallen below 0.1%. Thereafter, cooling was effected. The product was obtained as a colourless oil.

### Example 7.1

### Use of a Fluorinated Plasticizer

Analogously to the procedure in Example 5.1-5.6, 3.792 g of the polyol from Example 4.0, 2.500 g of Example 3.3, 2.500 g of the fluorinated plasticizer from Example 6.0, 0.1 g of CGI-909 (tetrabutylammonium tris(3-chloro-4-methylphenyl)(hexyl)borate), 0.015 g of 20 μm glass beads, 0.01 g of new methylene blue at 60° C. and 0.345 g of N-ethylpyrilidone are mixed so that a clear solution was obtained. Thereafter, cooling to 30° C. was effected, 0.702 g of Desmodur® XP 2410 was added and mixing was effected again. Finally, 0.006 g of Fomrez UL 28 was added and mixing was effected again briefly (by means of a speed mixer). The following holographic performance is obtained: Dn=0.0244/4 s exposure time/energy dose 18.1 mJ/cm²/12.0 μm calculated layer thickness.

### Example 7.2

### Use of a Fluorinated Plasticizer

Analogously to the procedure in Example 7.1, 3.370 g of the polyol from Example 4.0, 4.000 g of Example 3.3, 1.500 g of the fluorinated plasticizer from Example 6.0 and 0.624 g of Desmodur® XP 2410 are used. The other components are used in the same amount. The following holographic performance is obtained: Dn=0.0265/2 s exposure time/energy dose 9.11 mJ/cm²/18.0 μm calculated layer thickness.

### Example 8.1

### Preparation of a Writing Monomer not According to the Invention

### Phosphorothioyltris(oxybenzene-4,1-diylcarbamoyloxyethane-2,1-diyfltrisacrylate

In a 500 ml round-bottomed flask, 0.1 g of 2,6-di-tert-butyl-4-methylphenol, 0.05 g of dibutyltin dilaurate (Desmorapid Z, Bayer MaterialScience AG, Leverkusen, Germany) and 213.07 g of a 27% strength solution of tris(p-isocyanatophenyl)thiophosphate in ethyl acetate (Desmodur® RFE, product of Bayer MaterialScience AG, Leverkusen, Germany) were initially introduced and heated to 60° C. Thereafter, 42.37 g of 2-hydroxyethyl acrylate were added dropwise and the mixture was further kept at 60° C. until the isocyanate content had fallen below 0.1%. Thereafter, cooling was effected and the ethyl acetate was completely removed in vacuo. The product was obtained as a semicrystalline solid.

### Example 8.2

### Use of a Further Writing Monomer

Analogously to the procedure in Example 7.1, 5.901 g of the polyol from Example 4.0, 1.500 g of the writing monomer according to the invention from Example 3.2, 1.000 g of the writing monomer not according to the invention from Example 8.1 and 1.093 g of Desmodur® XP 2410 are used. The other components are used in the same amount. The following holographic performance is obtained. Dn=0.0061/4 s exposure time/energy dose 18.22 mJ/cm2/calculated layer thickness 25.0 μm.

### Example 8.3

### Use of a Further Plasticizer

Analogously to the procedure in Example 7.1, 4.636 g of the polyol from Example 4.0, 2.500 g of the writing monomer according to the invention from Example 3.2, 1.500 g of the plasticizer from Example 6.0 and 0.859 g of Desmodur® XP 2410 are used. The other components are used in the same amount. The following holographic performance is obtained. Dn=0.0060/4 s exposure time/energy dose 18.22 mJ/cm2/calculated layer thickness 15.0 μm.

### Example 8.4

### Use of a Further Writing Monomer and of a Fluorinated Plasticizer

Analogously to the procedure in Example 7.1, 4.636 g of the polyol from Example 4.0, 1.500 g of the writing monomer according to the invention from Example 3.2, 1.000 g of the writing monomer not according to the invention from Example 8.1, 1.500 g of the plasticizer from Example 6.0 and 0.859 g of Desmodur® XP 2410 are used. The other components are used in the same amount. The following holographic performance is obtained. Dn=0.0026/8 s exposure time/energy dose 36.45 mJ/cm2/calculated layer thickness 17.0 μm.

As shown in Table 4 and Examples 7.1, 7.2 and 8.2-8.4, the holographic media according to the invention have a good holographic performance. The index modulation is between 0.0026 and 0.0265. In addition, the preparation of the methacrylates according to the invention (Examples 1.1-3.3) can be carried out easily, in particular no distillation step is required.

1.-13. (canceled)

14. A methacrylate having the formulae (I) or (II) and mixtures thereof 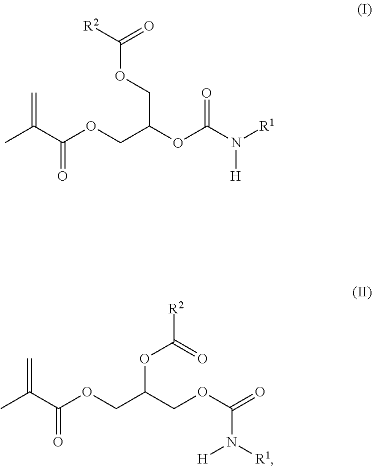
wherein R¹ and R², independently of one another, represent substituted phenyl radicals, or substituted or unsubstituted naphthyl radicals.

15. The methacrylate according to claim 14, wherein R¹ and/or R² comprise 6-24 C atoms, 0-5 S atoms and 0-5 halogen atoms.

16. The methacrylate according to claim 14, wherein R¹ and/or R² are substituted by thioether groups, phenyl groups and/or halogen atoms.

17. The methacrylate according to claim 14, wherein R¹ and/or R² represent naphthyl, 3-methylthiophenyl, 2-, 3- or 4-biphenyl, 2-bromophenyl.

18. A process for the preparation of the methacrylate according to claim 14, comprising reacting an aromatic acid of the formula R²—COOH with glycidyl methacrylate and subsequently reacting the product with an aromatic isocyanate of the formula R¹—NCO.

19. A photopolymer formulation comprising matrix polymers, writing monomers and photoinitiators, wherein the writing monomers comprise the methacrylate according to claim 14.

20. The photopolymer formulation according to claim 19, wherein the matrix polymers comprise polyurethanes.

21. The photopolymer formulation according to claim 19, wherein the photoinitiators comprise an anionic, cationic or neutral dye and a coinitiator.

22. The photopolymer formulation according to claims 19, wherein the photopolymer formulation further comprises urethanes as plasticizers, wherein the urethanes are optionally substituted by at least one fluorine atom.

23. The photopolymer formulation according to claim 22, wherein the urethanes have the formula (III) 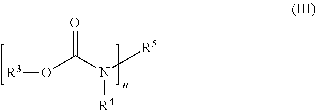
wherein
n is from 1 to 8 and
R³, R⁴, R⁵, independently of one another, represent hydrogen or linear, branched, cyclic or heterocyclic organic radicals which are unsubstituted or optionally also substituted by heteroatoms.

24. The photopolymer formulation according to claim 23, wherein at least one of the radicals R³, R⁴, R⁵ is substituted by at least one fluorine atom.

25. The photopolymer formulation according to claim 23, wherein R³ represents an organic radical having at least one fluorine atom.

26. The photopolymer formulation according to claim 19, wherein the writing monomers additionally comprise a polyfunctional writing monomer.

27. The photopolymer formulation according to claim 26, wherein the polyfunctional writing monomer comprises a polyfunctional acrylate.

28. The photopolymer formulation according to claim 27, wherein the polyfunctional acrylate has the formula (IV) 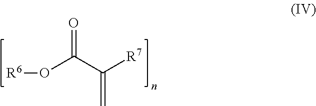
wherein
n is from 2 to 4 and
R⁶, R⁷, independently of one another, represent hydrogen or linear, branched, cyclic or heterocyclic organic radicals which are unsubstituted or optionally also substituted by heteroatoms.

29. An in-line hologram, off-axis hologram, full-aperture transfer hologram, white light transmission hologram, Denisyuk hologram, off-axis reflection hologram, edge-lit hologram and holographic stereogram formed from the photopolymer formulation according to claim 19.
